(19) United States
(12) Patent Application Publication
(10) Pub. No.: US 2003/0092090 A1
(43) Pub. Date: May 15, 2003

(54) Rapid prion-detection device, system, and test kit

(76) Inventors: Kiamars Hajizadeh, Buffalo Grove, IL (US); Zakir S. Murtaza, Arlington Heights, IL (US)

Correspondence Address: Wallenstein & Wagner, Ltd., 53rd Floor, 311 S. Wacker Drive, Chicago, IL, 60606-6622, US

(21) Appl. No.: 09/992,533

(22) Filed: Nov. 14, 2001

(51) Int. Cl. G01N 33/53; G01N 33/537; G01N 33/543; C12M 1/34

(52) U.S. Cl. 435/7.92; 435/287.2

(57) ABSTRACT

Test devices, systems, and test kits are provided for rapid detection with high specificity of the pathogenic form of prion protein responsible for neurodegenerative diseases affecting humans and animals, such as transmissible spongiform encephalopathy in bovine, sheep, and cats. The present invention is also useful for testing animal feedstock made from animal parts. Results are available in from about 0.5 to about 20 minutes and preferably from about 5 to about 10 minutes after the sample is introduced to the device and system. The devices, systems, and test kits employ proteinase-K to remove noninfectious prion protein from a biological sample, so that the sample may be analyzed by immunochromatography to determine the presence and concentration of pathogenic prion protein. Because the proteinase-K is immobilized on a solid support for in-situ removal of interfering components, the present invention obviates the need for subsequent extraction of the desired analyte. All aspects of the present invention are suitable for quantifying the minimal detectable amount of pathogenic prion protein in a biological sample. Moreover, the simplicity of sample preparation makes the present invention suitable for use in the field.

### TECHNICAL FIELD

This invention relates to a rapid diagnostic device, system, and test kit for testing for disease in animals and humans, and more particularly to a device, system, and test kit for detecting the pathogenic form of prion in biological fluids and tissues obtained from animals and humans suspected of having a prion-caused disease and in animal feedstock made from animal parts.

### BACKGROUND OF INVENTION

Humans and animals develop a variety of transmissible neurodegenerative disorders as a result of infection by prions—aberrant proteins that join bacteria, viruses, and viroids as infectious pathogens. Examples of prion diseases afflicting animals include scrapie in sheep and goats, and bovine spongiform encephalopathy (BSE) in cattle. Animals may contract a prion disease by consuming feed made from organs and other components from infected animals, such as cow udders and bone in the form of bone meal. Humans are subject to four prion diseases including kuru, Creutzfeldt-Jakob disease, Gerstmann-Strassler-Scheinker disease, and fatal familial insomnia. Humans may contract Creutzfeldt-Jakob disease by consuming beef, as an example, infected with prions.

A conformational change that occurs in the normal host prion protein causes prion diseases by converting the normal prion protein into an abnormal aggregate-forming pathogenic structure known as a prion. The pathogenic form of prion protein is designated as “PrP^{SC}”; the normal form is designated as “PrP^{C}.”

Detection of prions is difficult because of the poor solubility of prions in many biological buffers and the tenacity of its aggregates in resisting dissolution. As a result, the methodology used for analyzing prions is oftentimes time-intensive and complex. For example, hydrophilic-interaction chromatography has been used to purify the abnormal prion protein, followed by capillary electrophoresis immunoassay for detection. Schmerr and Jenny, Electrophoresis 19:409 (1998), cited in U.S. Pat. No. 6,150,172.

Despite these problems, however, various assays are known in the art for selectively detecting abnormal prion protein Among the immunoassays for determining prion protein are techniques such as radioimmunoassay, ELISA (enzyme-linked immunosorbant assay), immunoradiometric assays, gel diffusion precipitation reactions, immunodiffusion assays, in situ immunoassays (using colloidal gold, enzyme or radioisotope labels), Western blots, precipitation reactions, agglutination assays (e.g., gel agglutination assays and hemagglutination assays), complement fixation assays, immunofluorescence assays, protein A and protein G assays, and immunoelectrophoresis assays.

Immunochromatographic assays are known for their ability to analyze proteins. For example, U.S. Pat. No. 6,180,417, issued to Hajizadeh et al., discloses an immunochromatographic assay, featuring both “sandwich” and competitive formats. U.S. Pat. Nos. 4,703,017 issued to Campbell et al. and 5,591,645 issued to Rosentein use visible particles in immunochromatography test strips. The test strip and assay of these patents, however, do not provide for the extraction and rapid analysis of pathogenic prion protein.

In U.S. Pat. No. 6,214,565, Prusiner et al. disclose a time- and labor-intensive assay for isolating and detecting the infectious prion protein in materials from human, bovine, sheep, goat and other animals. The assay involves treating a homogenized sample with a protease to remove substantially all non-infectious prion protein. The prion in the treated sample is then crosslinked to a plastic support. The filter is next immersed and incubated in an antibody-containing solution, followed by removal of the unbound antibody. The immersion/incubation/antibody-removal step is repeated with a second solution containing an anti-Ig antibody, typically radiolabled. Results are determined by immunoblot detection, using X-ray film. Conservatively, the assay takes at least four hours to prepare the filter for immunoblot detection.

U.S. Pat. No. 6,150,172 issued to Schmerr et al. discloses a three-step method for extracting abnormal prion protein from homogenized biological material and analyzing the extracted protein with a chromatographic immunoassay. The extraction method includes incubating an aqueous preparation of the biological sample with a pre-measured amount of proteinase-K to digest the normal prion protein, isolating the pathogenic prion protein by mixing the pre-treated sample with an extraction solvent, and recovering the isolated pathogenic prion protein in the extraction solvent. Col. 4, lines 21-26. The method shortens the extraction time to 1 to 2 hours. Col. 9, lines 27-28.

Schmerr et al. disclose that the extraction solvent can then be applied directly to a support and assayed via immunochromatography. The following U.S. patents set forth examples of immunochromatographic assays, known in the art, that may be used for assaying the extraction solvent: U.S. Pat. Nos. 5,248,619; 5,451,504; 5,500,375; 5,624,809; and 5,658,801. Though the referenced method isolates and detects abnormal prion protein, it involves multiple steps and requires as much as two hours for merely extracting the analyte.

Thus, there exists a need for a device and simplified method for rapidly determining the presence and/or concentration of pathogenic prions in biological samples. There also exists a need for test devices and assays that are capable of detecting nanogram quantities of pathogenic prions, particularly, for, e.g., detecting bovine spongiform encephalopathy in animal carcasses in the meat-processing industry.

### SUMMARY OF THE INVENTION

The present invention is directed to devices, test systems, and test kits for determining the presence and concentration of pathogenic prion protein in a biological sample obtained from a human or an animal. Each aspect of the invention incorporates proteinase-K immobilized on a support to digest substantially all the non-pathogenic form of prion protein analyzed by immunochromatography.

A first aspect in accordance with the invention is an assaying device for detecting the presence of pathogenic prion protein in a biological sample and in material made from a biological sample. The device comprises a digestive pad having proteinase-K immobilized therein for removing nonpathogenic prion protein from the biological sample; a conjugate pad having a labeled first antibody of an antibody pair to the pathogenic prion protein; and a test strip having an immobilized second antibody of the antibody pair for producing a response indicative of the presence or concentration of the pathogenic prion protein. The conjugate pad is in fluid communication with the digestive pad and the test strip. The label on the first antibody in the conjugate pad is selected from latex beads, rod-shaped bodies coated with latex, particles comprising a dye, colloidal particles, metal particles, micro- and nano-particles, fluorescent compounds, chemiluminescent compounds, and magnetic beads.

In a second aspect in accordance with the invention, an assaying device is provided for detecting the presence of pathogenic prion protein in a biological sample and in material made therefrom. The device comprises proteinase-K immobilized on a support for digesting nonpathogenic prion protein in the biological sample; a conjugate pad impregnated with a labeled first antibody to the pathogenic prion protein for complexing with the pathogenic prion protein; and a test strip having a first end, a second end, and a second antibody to the pathogenic prion protein immobilized between the conjugate pad and the second end, such that the immobilized antibody produces a detectable change in the presence of the pathogenic prion protein. The conjugate pad is disposed between and in fluid communication with the proteinase support and the test strip.

In a third aspect of the present invention, a test system is provided for detecting pathogenic prion protein in animals or humans. The test system comprises: (a) proteinase-K immobilized on a support and suitable for removing nonpathogenic prion protein from a biological sample from the animal or the human; (b) a porous membrane for the sample substantially free of the nonpathogenic prion protein to migrate laterally therethrough by capillary action; and (c) a pair of antibodies specific to the pathogenic prion, one antibody being labeled antibody for complexing with the pathogenic prion protein, and the other antibody being immobilized on the membrane for capturing the labeled antibody complex and producing a corresponding response.

In a fourth aspect in accordance with the present invention, a test kit is provided for rapid detection of pathogenic prion in a biological sample and in a material made from a biological sample. The test kit has (a) a buffer for homogenizing a sample containing biological material obtained from an animal or a human; (b) proteinase-K immobilized on a support for removing nonpathogenic prion protein from the homogenized sample; (c) a porous membrane for the sample substantially free of the nonpathogenic prion protein to migrate laterally therethrough by capillary action; and (d) a pair of antibodies specific to the pathogenic prion. One antibody is an antibody for complexing with the pathogenic prion protein present in the sample, and the other antibody is immobilized on the membrane for capturing the labeled antibody complex and producing a corresponding response.

The buffer includes at least one emulsifier or surfactant, casein, at least one polysaccharide, albumin, and a sufficient quantity of water to form a mixture. The at least one emulsifier or surfactant is typically octoxynol, nonoxynol, polyglycol ether, polyoxythylene (10) isooctylphenyl ether, sodium dodecyl sulfate (SDS), and sodium deoxycholate. The at least one polysaccharide is, e.g., sucrose, mannose, trehalose, or maltose.

In a fifth aspect in accordance with the invention, a test kit is provided for rapid detection of pathogenic prion protein. The test kit includes (a) a buffer for extracting prion protein from a sample containing biological material obtained from from a vertebrate; (b) proteinase-K immobilized in a digestive pad; (c) a test strip having an immobilized antibody of an antibody pair to the pathogenic prion protein; and (d) a labeled antibody to the pathogenic prion protein for producing a readable response indicative of the presence or concentration of the pathogenic prion protein. The test strip is in fluid communication with the digestive pad and has pores of a size sufficient to allow the labeled antibody to migrate therethrough.

In this aspect of the invention, as homogenized enzyme-treated sample flows through the test strip, the labeled antibody and the immobilized antibody each bind to a specific epitope of the pathogenic prion protein to produce the response.

All aspects of the present invention produce results within from about 0.5 to about 20 minutes after the homogenized sample is introduced to the test strip or porous membrane, and preferably within about 5 to about 10 minutes. The device, system, and test kit have application in analyzing prion protein responsible for a number of prion-caused diseases in both animals and humans, such as transmissible spongiform encephalopathy (TSE) in bovine, sheep, and goats and Creutzfeldt-Jakob-disease (CJD) in humans. Because of their simplicity of sample preparation and analysis, the device, system, and test kit are especially suitable for use in the field.

### BRIEF DESCRIPTION OF THE DRAWINGS

To understand the present invention, it will now be described by way of example, with reference to the accompanying drawings in which:

FIG. 1 is a side perspective view of one embodiment of a test device in accordance with the teachings of the present invention;

FIG. 2 is a side perspective view of another embodiment of a test device in accordance with the invention;

FIG. 3 is a top schematic view of another embodiment of a test device made in accordance with one aspect of the invention; and,

FIG. 4 is a side perspective view of still another embodiment of the test device made in accordance with the invention.

### DETAILED DESCRIPTION OF THE INVENTION

While this invention is susceptible of embodiments in many different forms, preferred embodiments of the invention are illustrated in the drawings and described in detail herein, with the understanding that the present disclosure is to be considered as an exemplification of the principles of the invention and is not intended to limit the broad aspect of the invention to the embodiments illustrated.

The present invention is directed to testing devices, systems, and methods that utilize immunochromatography for determining the presence and concentration of pathogenic prion protein in a biological sample. The present invention utilizes immobilized proteinase-K (PK) enzyme for in-situ removal of interfering components. The devices, systems, and methods are suitable for quantifying the minimal detectable amount of pathogenic prion protein in a biological sample. Moreover, the rapid detection of pathogenic prion protein with high specificity, combined with the simplicity of preparing the sample, makes the present invention suitable for use in the field.

The test devices, systems, and methods may be used for rapid detection of prion diseases such as scrapie and spongiform encephalopathy in bovine, sheep, cats, and other animals. Additionally, the devices, systems, and methods may be used by the medical community for analysis of human tissue for kuru, Creutzfeldt-Jakob disease, Gerstmann-Straussler-Scheinker disease and fatal familial insomnia.

Throughout this application, the following terms have the meanings set forth below. “Biological material” or “biological sample” refers to fluid, tissue, and organs extracted from vertebrates, such as brain tissue, whole blood, serum, plasma, saliva, urine, and cerebral spinal fluid. Herein, these terms also refer to materials made from animal fluids, tissues or organs, such as animal feed.

“Label” refers to a component or “tag” that is attached covalently to a protein of choice. The label could be from a number of detectable groups such as enzymes, visible particles, nanoparticles, and fluorescent components, as examples.

“PrP^{C}” refers to the nonpathogenic form of prion protein, which is enzymatically removed from the biological sample.

“PrP^{SC}” refers to the pathogenic prion protein which is the analyte in the methods of this invention.

Sample Preparation

The present methods, test devices, and systems are used with a biological material extracted from an animal or human. Samples of brain tissue, including organs, are extracted post-mortem; but other samples—such as urine, whole blood, serum, and plasma—may be obtained from the live animal or human.

The biological sample is homogenized with a suitable quantity of buffer formulated to optimize the extraction of prion protein into the buffer medium. Homogenization may be accomplished by any technique known in the art, including, e.g., shaking the biological material with weights, vortexing the material, digesting the same with ultrasonic waves, or comminuting the sample in a homogenizer. Preferably, however, homogenization is conducted by either vortexing or shaking the material with weights.

The buffer does not have organic solvents. Typically, the buffer is an aqueous solution formulated to have an ionic strength of from about 200 to about 400 mM to facilitate prion extraction from the sample. The buffer comprises at least one emulsifier or surfactant, casein, at least one polysaccharide such as a sugar, albumin such as bovine serum albumin (BSA), and a sufficient quantity of water to form a mixture. Typically, the emulsifiers include at least one emulsifier or surfactant such as octoxynol (e.g., IGEPAL®), nonoxynol, polyglycol ether (e.g., Tergitol® NP), polyoxythylene (10) isooctylphenyl ether, sodium dodecyl sulfate (SDS), or sodium deoxycholate, as examples. A preservative may be used; e.g., ethylene-diamine-tetraacetic acid (EDTA) and sodium azide. The polysaccharides include at least one of sucrose, mannose, trehalose, maltose, and other suitable polysaccharides, as examples, in an amount sufficient to yield a molar concentration ranging from about 60 to about 80 mM. Additionally, the buffer may contain a denaturing compound such as guanidine hydrochloride, urea, and guanidine isothiocyanate. The buffer may also contain a zwitterionic buffering salt, such as 4-(2-hydroxyethyl)-1-piperazineethane-sulfonic acid (HEPES), used at a concentration ranging from about 1.5 to about 5%, by weight, to maintain the integrity of the solid support for the enzyme used downstream in the analysis. The total concentration of the emulsifiers and surfactants ranges from about 0.05 to about 5%, by weight of the buffer, and the casein generally ranges from about 10 to about 40%, by weight of the buffer. The total concentration of the polysacccharides ranges from about 0.1 to about 30%, by weight of the buffer. The albumin is typically used at a concentration ranging from about 0.5 to about 4%, by weight of the buffer. The zwitterionic buffering agent may used at a concentration ranging from about 2 to about 5%, by weight. The denaturing agent may be present at a concentration ranging from about 0.1 to about 1 M.

The total concentration of the emulsifiers and surfactants ranges from about 0.05 to about 5%, by weight of the buffer, and the casein generally ranges from about 10 to about 40%, by weight of the buffer. The total concentration of the polysacccharides ranges from about 0.1 to about 30%, by weight of the buffer. The albumin is typically used at a concentration ranging from about 0.5 to about 4%, by weight of the buffer. The zwitterionic buffering agent may used at a concentration ranging from about 2 to about 5%, by weight. The denaturing agent may be present at a concentration ranging from about 0.1 to about 1 M.

An example of a suitable buffer is shown in Table 1.

TABLE 1
Example of a Buffer Formulation for Extracting Prion Protein.

| Buffer constituent | Concentration (wt %) |
|---|---|
| octoxynol | 0.1 |
| casein | 40.0 |
| HEPES | 3.0 |
| EDTA | 0.2 |
| trehalose | 0.1 |
| sucrose | 18.5 |
| BSA | 1.0 |
| NaCl | 1.5 |
| sodium deoxycholate | 0.5 |
| SDS | 0.4 |
| water | 34.7 |

The homogenate is prepared by homogenizing the biological sample with buffer in a weight/volume ratio of sample (mg) to buffer (ml) ranging from about 2:1000 to about 200:1000, and preferably from about 5:1000 to about 100:1000. Most preferably, the ratio of sample (mg) to buffer (ml) is about 30:1000 to about 70:1000.

A. Test Device

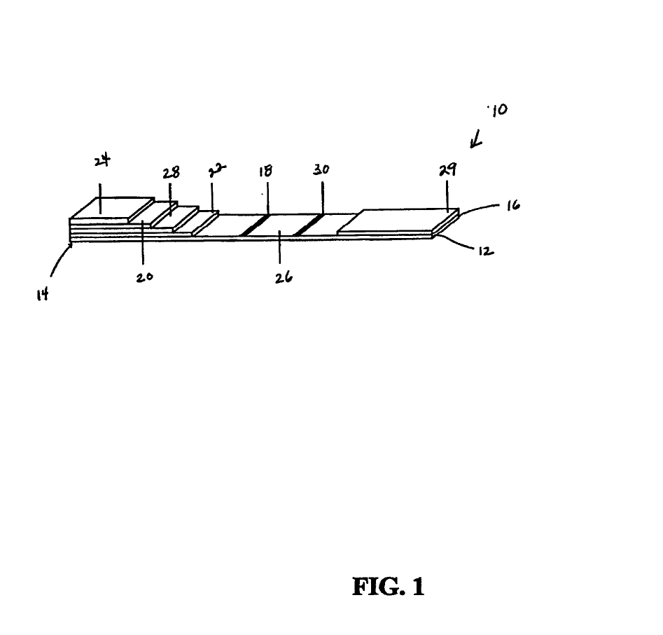

Shown in FIG. 1 is a test device 10 of a first embodiment. The test device 10 utilizes a pair of antibodies specific to PrP^{SC}. These include (1) a labeled antibody that “detects” the PrP^{Sc} and (2) an immobilized antibody that “captures” the prion protein-antibody-label complex to form a “sandwich.” Briefly, in this invention, a homogenized sample of a biological material is introduced to the test device. In the preferred embodiment, the sample first moves through a zone containing immobilized proteinase-K (PK), which digests the nonpathogenic prion protein, leaving the PrP^{SC} for analysis. The proteinase-K is immobilized to a solid support. The removal of the normal prion protein minimizes sample interference and results in a higher specificity for the analyte. As the treated sample moves through the test device, it encounters the first specific antibody conjugated to a label and affixed to a portion of the test device. In one embodiment, the label is a colored latex bead.

The fluid in the homogenized sample re-suspends the antibody-label conjugate so it is free to move through the device. As the antibody-label conjugate moves through the membrane, the labeled antibody binds to a particular epitope of the PrP^{SC} to form a prion protein-antibody-label complex. Via capillary force, the labeled complex migrates through the porous membrane of the device until it reaches the second specific antibody. This antibody is immobilized on the membrane, typically in the form of a band or stripe. The second antibody binds to the second epitope of the PrP^{SC} to which it is specific, resulting in the analyte becoming “sandwiched” between the two antibodies. The resulting “sandwiched” PrP^{SC} produces a detectable change in the membrane, such as the formation of a colored test line, which indicates a positive result. In the absence of antigen, no “sandwich” complex forms and no test line appears.

In an alternative embodiment, the test strip may include more than one “capture” antibody, each applied in a separate test line with each test line being specific to a different prion disease, so that the test device may be used for screening purposes.

The test device 10 includes a test strip 12 having an anterior end 14, a distal end 16, and a “test line” 18 therebetween. The test strip 12 comprises an absorbent material having pores (not shown) ranging from about 10 to about 1000 microns, and preferably from about 10 to about 100 microns. The pores are generally of a size sufficient to allow the homogenized sample, including the re-suspended labeled antibody and conjugates formed by the labeled antibody binding with prion proteins, to migrate laterally through the test strip 12 toward the test line 18.

The test strip itself has at least one layer of absorbent material. Suitable materials include at least one of, e.g., nitrocellulose, cellulose, glass fiber, bonded glass fiber, polyesters, nylon, polyethylsulphone, and other materials having absorbent properties, all of which allow an aqueous sample applied at one end of the test strip to migrate to the opposite end by capillary action.

Although FIG. 1 shows the nitrocellulose membrane or test strip 12 as being rectangular in shape, the test strip, of course, may have virtually any shape that allows an analyte to travel from a point where the sample is introduced to a point where the analyte is detected. Accordingly, the test strip may be square, triangular, circular, or octagonal, or any other suitable shape.

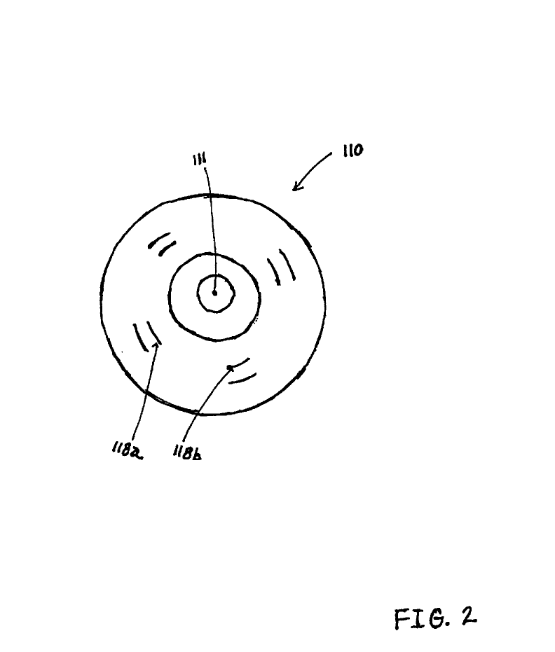

FIG. 2 shows the test device 110 having a circular configuration, with the immobilized antibody being affixed at a predetermined distance from the sample-introduction site 111. The embodiment shown in FIG. 2 has antibodies for two prion diseases and thus allows the respective pathogenic prion proteins to be analyzed for these in the same test device. Test lines 118a,b each have immobilized antibodies corresponding to the pathogenic prion protein of a different prion disease which allows the device to be used as a diagnostic tool. Any of the test devices, irrespective of their shape, may be used to analyze more than one prion disease at the same time.

In a preferred embodiment, the test strip 12 is affixed to a strip support 13 of a sufficiently rigid, impervious and non-reactive material such as polystyrene, polyvinyl chloride, and polyethylene terephthalates. Typically, the strip support is hydrophobic in nature to ensure that the maximum amount of test sample is directed for analysis. In a preferred embodiment, the strip support includes at least one layer of an impervious material.

In yet another embodiment, the entire test strip, and ancillary components described below, may be at least partially encased in a device holder for protecting the device from the environment. This form of the test device is best suited for use in more demanding test environments such as slaughterhouses.

At or near the anterior end 14 of the test strip 12, shown in FIG. 1, is a digestive pad 20 comprising immobilized proteinase-K for digesting nonpathogenic prion protein present in the homogenized biological sample. The digestive pad 20 is generally an absorbent material such as gauze but may comprise other suitable materials such as a plastic filter bed in glass fiber, polyester, and plastic bonded glass fiber, as examples.

The proteinase-K may be bound covalently to the digestive pad or conjugated to a solid support (not shown) impregnated in the digestive pad. The solid support may be, e.g., latex beads, rod-shaped bodies coated with latex, micro- or nanoparticles, beads coated with a dye or a fluorescent or chemiluminescent compound, or a porous membrane pad. Additionally, the proteinase-K may be incorporated into the digestive pad in a gelled substance contained therein. The latex beads in the digestive pad have an average diameter of from about 1 to about 10 microns.

The amount of enzyme on the support medium usually ranges from about 30 μg to about 400 μg and preferably from about 100 μg to about 350 μg. The amount of enzyme is sufficient to substantially digest all PrP^{c} present in the sample; typically, this amount is at least 30 units of enzyme per mg of all protein present in the sample. The enzyme treatment is conducted for a time and at a temperature sufficient for the proteinase-K to digest the nonpathogenic prion protein. Generally, digestion is completed in about 2 to about 15 minutes depending upon the amount of prion present, when conducted at temperatures ranging from about 25° C. to about 60° C.

A conjugate pad 22 is disposed between the digestive pad 20 and the test strip 12, generally near the anterior end 14 of the test strip 12, and is impregnated with a label—typically a particulate—conjugated to one of the antibodies specific to the PrP^{SC}. As noted above, the particulates function as labels on the antibodies, allowing easy detection downstream on the nitrocellulose membrane. Suitable particulates for conjugation with the antibody include latex beads, rod-shaped bodies coated with latex, particles comprising a dye, colloidal particles, metal particles, micro- and nanoparticles, fluorescent compounds, chemiluminescent compounds, and magnetic beads, as examples. In one embodiment, the particulates are latex beads filled or coated with a dye, such as blue latex beads. The latex beads typically have an average diameter of from about 50 to about 500 nanometers and preferably from about 100 to about 350 nanometers. The magnetic beads have an average diameter of from about 50 to about 350 nanometers and preferably from about 100 to about 300 nanometers.

The conjugate pad comprises any absorbent material or suitable support for the labeled antibodies, such as at least one of plastic filter bed in glass fiber, polyester, plastic bonded glass fiber, nonwoven polymeric material, as examples. The conjugate pad lies in direct fluid communication with the test strip.

An alternative embodiment includes a filter pad 24 in fluid communication with the digestive pad 20, opposite the conjugate pad 22. A homogenized sample may be applied to the filter pad 24, an absorbent pad of a material that receives the fluid sample and allows it to flow into the conjugate pad 22. The filter pad 24 may also function to remove larger particles that may interfere with the assay. The filter pad 24 may comprise any suitable material such as gauze, cellulose, cellulose acetate, other polyesters, and other porous membranes, for example. Alternatively, the sample may be filtered in a separate step prior to its introduction to the digestive pad.

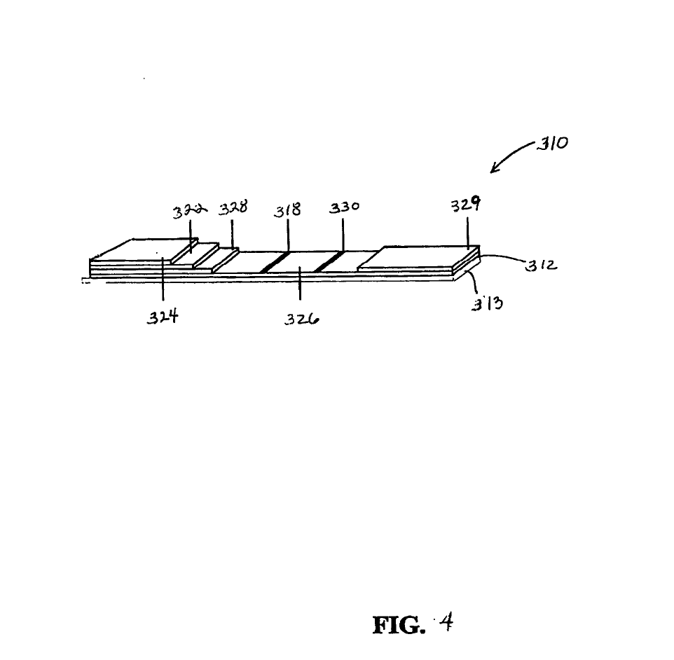

The test device 10 also has a detection region 26 (shown in FIG. 1 and designated by reference numeral “326” in FIG. 4) where the user may view the test result. The detection region 26 includes the test line 18 (shown as “318” in FIG. 4) and the control line 30 (shown as “330” in FIG. 4), when incorporated into the device.

As shown in FIG. 1, the three pads may be layered one atop the other at or near the anterior end, such that the filter pad 24 is the pad farthest from the test strip 12, the conjugate pad 22 is adjacent and substantially aligned with the test strip 12, and the digestive pad 20 is between the filter pad and the conjugate pad.

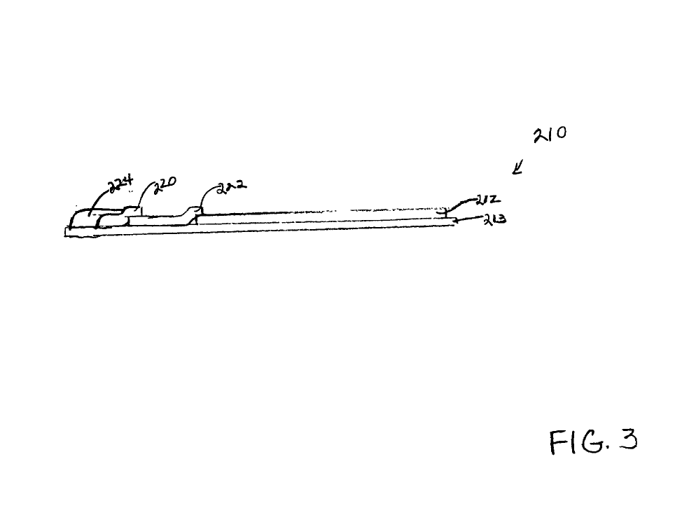

In a preferred embodiment of device 210, shown in FIG. 3, the pads lie substantially in the same plane, staggered with respect to each other, so that only a portion of one pad is in contact with a portion of an adjacent pad. Typically, the contact portion is in the form of an overlay between adjacent pads, such that the overlay, as well as the overlay between the test strip 212 and the adjacent pad, ranges from about 0.5 to about 5 millimeters and preferably from about 1 to about 2 millimeters. Shown in FIG. 3 are filter pad 224, digestive pad 220, and conjugate pad 222. In the preferred embodiment, at least a portion of each pad and the test strip 212 is adhered to the support 213. The selection, shape, size, and positioning of the pads with respect to each other and the test strip 212 may be optimized as needed. In one embodiment, the pad may be distinct portions of one composite test pad.

The order of the pads may be substantially as set forth above; e.g., the filter pad being the farthest from the detection region, followed by the digestive pad, and then, the conjugate pad or any other suitable configuration. Each pad may have an outer edge generally corresponding in size and shape with that of the other pads, although other configurations are encompassed within the scope of this invention.

An additional pad may be needed to separate digestive pad from the conjugate pad. In another embodiment of the invention, the test strip may have a single pad impregnated with PK enzyme, serving both as the digestive pad and the filter pad. Though optional, a spacer pad 228 may be disposed between the digestive pad 220 and the conjugate pad 222 to allow for more complete digestion of the normal prion before it reaches the conjugate pad.

As shown in FIG. 1, in the detection region 26 lies the second antibody specific to the PrP^{SC}, typically immobilized on the membrane in the form of the “test line” or stripe. Alternatively, the antibody may be affixed in any suitable configuration that allows the test result to be viewed, or otherwise read, visually or by instrumentation. In another embodiment, the response may be compared against known responses or a standard curve to determine the concentration of the analyte.

In another embodiment, as shown in FIG. 1, the test device 10 includes a wicking pad 29 at the distal end of the test strip 12. The wicking pad 29 promotes the capillary flow of the homogenized fluid sample through the test strip by “drawing” the fluid sample to the distal end.

Generally, the amount of sample introduced to the test device is in the microliter range, typically from about 5 to about 500 microliters and preferably from about 75 to about 150 microliters.

In yet another embodiment, the test device includes a control line (shown by reference numeral “30” in FIG. 1 and “130” in FIG. 3) for indicating that the test is working properly. The control line, in fixed relation to the test line, comprises an antibody to the labeled antibody, such as immunoglobulin antibody, which binds with labeled antibody to produce a visually detectable line. Alternatively, the control line may be an antibody that binds with a secondary label on the particulate or bead, such as a protein or biotin-avidin binding sites.

The test line is permanent, but it could become visually more pronounced over time. Preferably, the test result is read within from about 2 to about 10 minutes from the time the homogenized sample is applied to the test strip.

The present invention allows pathogenic prion protein to be detected within from about 0.5 to about 20 minutes after the sample is introduced to the test device and preferably within from about 5 to about 10 minutes. The invention allows substantially real-time reading of the results on the test strip so that a test result is available almost instantaneously. Therefore, the preferred embodiment of this invention employs enzyme digestion within the test device so that the sample is subjected to only one labor-intensive step; i.e., homogenization. However, when the enzyme pre-treatment is conducted separately from the test strip, detection via the immunochromatographic phase may yield a readable result in from about 1 to about 5 minutes after sample introduction and preferably from about 2 to about 10 minutes, depending upon the concentration of normal prion protein to be denatured.

B. Test System

Another aspect of the invention is a testing system for detecting PrP^{SC} in a biological sample and material containing or made from a biological sample. In this aspect of the invention, the testing system comprises (a) proteinase-K immobilized on a support external to the test strip, for denaturing the nonpathogenic form of prion protein in a separate wet analysis conducted prior to introducing the homogenized sample to the test strip; and (b) a test strip that analyzes the enzymatically treated sample for the presence and concentration of PrP^{SC}. Shown in FIG. 4 is a test system device 310 suitable for use in this aspect of the invention. The test system is used with sample prepared as described above.

The test system and its operation are as described above for the device that performs both enzyme treatment and the assay. Components in FIG. 4 are similar to those in FIG. 1 and are represented by numbers in the 300 series. As shown, the test system device 310 has an impervious strip support 313 that is suitable for use in this aspect of the invention. Test device 310 includes a conjugate pad 322, a detection region 326, and a test line 318. Optionally, the test device may also include one or more of a filter pad 324, a spacer pad 328, and a wicking pad 329. Additionally, the test strip or membrane may incorporate a control line 330, described above, for determining whether the test is operating correctly. In this aspect of the invention, the support having the immobilized enzyme separate from the test strip displaces the digestive pad.

This aspect of the invention has application, e.g., when the prion must be heated in order to be digested and the proteinase-K treatment cannot be performed in real time without heating.

This aspect of the invention includes several embodiments. In this aspect, the support may be within, e.g., a beaker, a flask, a test tube, a cuvette, or any suitable container that may accommodate the support. In one embodiment, the support comprises magnetic beads. In an alternative embodiment, the support comprises, e.g., latex supports, filter tips, colloidal particles, microcrystalline particles, conjugate supports, plastic surfaces, and glass surfaces. The latex supports include, e.g., latex beads and latex-coated particles that may be of any shape. The amount of enzyme on the support medium ranges from about 30 μg to about 400 μg and preferably from about 100 μg to about 350 μg. The enzyme is used in an amount sufficient to substantially digest all PrP^{c} present in the sample; i.e., at least 30 units of enzyme per mg of all protein present in the sample.

When the sample is mixed with the support in, e.g., a test tube or a beaker, enzymatic digestion of the nonpathogenic prion protein is completed within about 15 minutes. Digestion is typically conducted at temperatures ranging from about 25° C. to about 60° C.

After digestion, the magnetic beads are separated from the mixture with a magnet rack or other suitable device, leaving a supernatant. Other forms of the solid support are removed from the treated sample by in-line filtration or any other suitable method. The supernatant is then applied to the test strip, without requiring further extraction of the prion analyte, for detecting and quantifying the PrP^{SC}. As described above, in the presence of PrP^{SC}, the test strip undergoes a detectable change, indicative of a positive result.

C. Test Kit

Another aspect of the invention is a test kit for rapidly detecting pathogenic prion in a biological sample from an animal or a human. The test kit produces results in from about 0.5 to about 20 minutes from the time the sample is introduced to the porous membrane or test strip.

A first embodiment comprises: (a) a buffer, described above, for homogenizing a biological sample obtained from an animal or a human to extract the prion protein; (b) proteinase-K immobilized on a support and suitable for digesting nonpathogenic prion protein present in the homogenized sample; (c) a porous membrane for the sample substantially free of the nonpathogenic prion protein to migrate laterally therethrough by capillary action; and (d) a pair of antibodies specific to the pathogenic prion. One of the antibodies, a labeled antibody, detects the pathogenic prion protein by complexing with the pathogenic prion protein at a specific epitope on the protein. The other antibody is immobilized on the membrane for capturing the labeled antibody complex by binding with a second epitope on the protein. The binding of the two antibodies to their respective epitopes produces a detectable response in the membrane.

In the first embodiment of the test kit, the support for the proteinase-K is external to the porous membrane. In this instance, the support for the proteinase-K is typically magnetic beads, latex supports, filter tips, colloidal particles, conjugate supports, plastic surfaces, or glass surfaces.

In a second embodiment of the test kit, the support for the proteinase-K is in a pad that communicates with the test strip. The second embodiment includes (a) a buffer, described above; (b) proteinase-K immobilized in a digestive pad for digesting nonpathogenic prion protein from the homogenized biological sample; (c) a test strip having an immobilized antibody to the pathogenic prion protein; and (d) a conjugate pad having a labeled antibody to the pathogenic prion protein. The conjugate pad is between the digestive pad and the test strip.

In both embodiments, the proteinase-K, the antibodies, the antibody labels, the membranes, the control line, and their mode of operation are substantially as described above. Alternatively, the proteinase-K is present in a gelled substance within the digestive pad. The amount of proteinase-K is sufficient to substantially digest all protein in the sample and typically ranges from about 30 μg to about 400 μg and preferably from about 100 μg to about 350 μg.

The buffer is formulated as described above. It includes at least one emulsifier or surfactant, casein, at least one polysaccharide, albumin such as bovine serum albumin, and a sufficient quantity of water to form a mixture. These constituents are as described above.

As with the test device and system, the test kit enjoys a simplicity of sample preparation. It allows the enzyme-treated homogenate to be applied to the porous membrane for immunochromatographic analysis, without requiring additional labor-intensive prion-extraction steps. Results are produced within from about 0.5 to about 20 minutes, and preferably within from about 5 to about 10 minutes, after the sample is introduced to the test strip. All aspects of the present invention are useful for testing biological fluids, tissue, and organs and materials containing such constituents; e.g., animal feed.

While the specific embodiments have been illustrated and described, numerous modifications come to mind without significantly departing from the spirit of the invention and the scope of protection is only limited by the scope of the accompanying claims.

We claim:

1. An assaying device for detecting the presence of pathogenic prion protein in a biological sample and in material made from a biological sample, comprising:
a digestive pad having proteinase-K immobilized therein for removing nonpathogenic prion protein from the biological sample;
a conjugate pad having a labeled first antibody of an antibody pair to pathogenic prion protein; the conjugate pad being in fluid communication with the digestive pad; and,
a test strip having an immobilized second antibody of the antibody pair for producing a response indicative of the presence or concentration of the pathogenic prion protein; the test strip being in fluid communication with the conjugate pad.

2. The device of claim 1 wherein the test strip has pores of a diameter sufficient to allow the labeled first antibody to migrate laterally through the test strip toward the immobilized antibody.

3. The device of claim 1 wherein the proteinase-K is bound covalently to the digestive pad.

4. The device of claim 1 wherein the proteinase-K is conjugated to components impregnated in the digestive pad.

5. The device of claim 1 wherein the proteinase-K is immobilized on a solid support selected from latex beads, rod-shaped bodies coated with latex, micro- or nanoparticles, and a porous membrane pad.

6. The device of claim 1 wherein the proteinase-K is in a gelled substance contained in the digestive pad.

7. The device of claim 1 wherein the amount of immobilized proteinase K in the digestive pad is sufficient to substantially digest all protein in the sample.

8. The device of claim 4 wherein the support comprises latex beads having an average diameter of from about 1 to about 10 microns.

9. The device of claim 1 wherein the amount of immobilized enzyme in the digestive pad ranges from about 30 μg to about 400 μg.

10. The device of claim 1 wherein the amount of immobilized enzyme in the digestive pad ranges from about 100 μg to about 350 μg.

11. The device of claim 1 wherein the labeled first antibody has a label selected from latex beads, rod-shaped bodies coated with latex, particles comprising a dye, colloidal particles, metal particles, micro- and nanoparticles, fluorescent compounds, chemiluminescent compounds, and magnetic beads.

12. The device of claim 1 wherein the labeled antibody has a colored label.

13. The device of claim 1 wherein the antibodies are each specific for a particular epitope of the pathogenic prion protein.

14. The device of claim 1 wherein the digestive pad and the conjugate pad lie adjacent each other in substantially the same plane.

15. The device of claim 1 further comprising a spacer pad between the digestive pad and the conjugate pad to allow for longer digestion of the nonpathogenic prion protein.

16. The device of claim 1 wherein the digestive pad and the conjugate pad comprise separate portions of a single pad.

17. The device of claim 1 wherein the digestive pad comprises at least one material selected from the group consisting of gauze, cellulose, cellulose acetate, polyesters, and porous materials.

18. The device of claim 1 wherein the conjugate pad comprises at least one of plastic filter bed in glass filter, polyester, plastic bonded glass fiber, and nonwoven polymeric materials.

19. The device of claim 1 wherein the test strip comprises at least one material selected from nitrocellulose, cellulose, glass fiber, bonded glass fiber, polyesters, nylon, and polyethylsulphone.

20. The device of claim 1 wherein each of the digestive pad, the conjugate pad, and the test strip are distinct portions of one composite test pad.

21. The device of claim 1 further comprising a control line for confirming the device is working, the control line comprising an antibody to the labeled first antibody.

22. An assaying device for detecting the presence of pathogenic prion protein in a biological sample and in material made therefrom comprising:
proteinase-K immobilized on a support;
a conjugate pad impregnated with a labeled first antibody to the pathogenic prion protein for complexing with the pathogenic prion protein; and,
a test strip having a first end, a second end, and an immobilized second antibody to the pathogenic prion protein immobilized between the conjugate pad and the second end, such that the immobilized antibody produces a detectable change in the presence of the pathogenic prion protein;
the conjugate pad is disposed between and in fluid communication with the proteinase support and the test strip.

23. The device of claim 22 wherein the proteinase-K support is one of a digestive pad or components impregnated in the digestive pad.

24. The device of claim 22 wherein the proteinase-K is immobilized on a solid support selected from latex beads, rod-shaped bodies coated with latex, micro- or nanoparticles, and a porous membrane pad.

25. The device of claim 22 wherein the proteinase-K is in a gelled substance contained in a digestive pad.

26. The device of claim 22 wherein the amount of proteinase-K immobilized in the digestive pad is sufficient to substantially digest all protein in the sample.

27. The device of claim 24 wherein the support is of a size too large to move through pores in the test strip.

28. The device of claim 22 wherein the amount of immobilized proteinase-K ranges from about 30 μg to about 400 μg.

29. The device of claim 22 wherein the amount of immobilized proteinase-K ranges from about 100 μg to about 350 μg.

30. The device of claim 22 wherein the label on the first antibody is selected from latex beads, rod-shaped bodies coated with latex, particles comprising a dye, colloidal particles, metal particles, micro- and nanoparticles, fluorescent compounds, chemiluminescent compounds, and magnetic beads.

31. The device of claim 22 wherein the labeled antibody has a colored label.

32. A test system for detecting pathogenic prion protein in animals or humans comprising:
(a) proteinase-K immobilized on a support;
(b) a porous membrane for a sample substantially free of the nonpathogenic prion protein to migrate laterally therethrough by capillary action; and,
(c) a pair of antibodies specific to the pathogenic prion, one antibody being a labeled antibody for complexing with pathogenic prion protein, and the other antibody being immobilized on the membrane for capturing the labeled antibody complex and producing a corresponding response in the test strip.

33. The system of claim 32 wherein the support is separate from the test strip.

34. The system of claim 32 wherein the support for the proteinase-K is selected from the group consisting of magnetic beads, latex supports, filter tips, microcrystalline particles, colloidal particles, conjugate supports, plastic surfaces, and glass surfaces.

35. The system of claim 32 wherein the proteinase-K is covalently bound to the support.

36. The system of claim 32 wherein the proteinase K is present on the support in an amount sufficient to substantially digest all protein in the sample.

37. The system of claim 32 wherein the proteinase-K is present on the support in an amount ranging from about 30 μg to about 400 μg.

38. The system of claim 32 wherein the proteinase-K is present on the support in an amount ranging from about 100 μg to about 350 μg.

39. The system of claim 32 wherein the membrane has pores of a diameter sufficient to allow the labeled antibody complex to migrate laterally therethrough toward the immobilized antibody.

40. The system of claim 32 wherein the labeled antibody is in a conjugate pad in fluid communication with the membrane.

41. The system of claim 32 wherein the labeled antibody has a colored label.

42. The system of claim 32 wherein the labeled antibody has a label selected from latex beads, rod-shaped bodies coated with latex, particles comprising a dye, colloidal particles, metal particles, micro- and nanoparticles, fluorescent compounds, chemiluminescent compounds, and magnetic beads.

43. The system of claim 42 wherein the label is a latex bead having an average diameter of from about 50 to about 500 nanometers.

44. The system of claim 32 wherein the membrane comprises at least one material selected from nitrocellulose, cellulose, glass fiber, bonded glass fiber, polyesters, nylon, and polyethylsulphone.

45. The system of claim 32 further comprising a control line for confirming the system is operating properly, the control line comprising an antibody to the labeled antibody.

46. A test kit for rapid detection of pathogenic prion in a sample containing a biological material obtained from an animal or a human, comprising:
(a) a buffer for homogenizing a sample containing biological material obtained from an animal or a human;
(b) proteinase-K immobilized on a support for removing nonpathogenic prion protein from the homogenized sample;
(c) a porous membrane for the sample substantially free of the nonpathogenic prion protein to migrate laterally by capillary action; and
(d) a pair of antibodies specific to the pathogenic prion, one antibody being a labeled antibody for complexing with the pathogenic prion protein present in the sample, and the other antibody being immobilized on the membrane for capturing the labeled antibody complex and producing a corresponding response.

47. The test kit of claim 46 wherein the buffer comprises at least one emulsifier or surfactant, casein, at least one polysaccharide, salt, albumin, and a sufficient quantity of water to form a mixture.

48. The test kit of claim 47 wherein the at least one emulsifier or surfactant in the buffer is selected from octoxynol, nonoxynol, polyglycol ether, polyoxythylene (10) isooctylphenyl ether, sodium dodecyl sulfate, and sodium deoxycholate.

49. The test kit of claim 47 wherein the at least one polysaccharide in the buffer is selected from sucrose, mannose, trehalose, and maltose.

50. The test kit of claim 47 wherein the buffer comprises a denaturing agent.

51. The test kit of claim 46 wherein the buffer has an ionic strength of from about 200 to about 400 mM.

52. The test kit of claim 46 wherein the support for the proteinase-K is external to the membrane.

53. The test kit of claim 52 wherein the support for the proteinase-K is selected from the group consisting of magnetic beads, latex supports, filter tips, microcrystalline particles, colloidal particles, conjugate supports, plastic surfaces, and glass surfaces.

54. The test kit of claim 46 wherein the proteinase-K immobilized on the support is present in an amount ranging from about 30 μg to about 400 μg.

55. The test kit of claim 46 wherein the proteinase-K immobilized on the support is present in an amount ranging from about 100 μg to about 350 μg.

56. The test kit of claim 46 wherein the labeled antibody has a colored label.

57. The test kit of claim 46 wherein the labeled antibody has a label selected from latex beads, rod-shaped bodies coated with latex, particles comprising a dye, colloidal particles, metal particles, micro- and nanoparticles, fluorescent compounds, chemiluminescent compounds, and magnetic beads.

58. The test kit of claim 46 wherein the label on the antibody is a latex bead having an average diameter of from about 50 to about 500 nanometers.

59. The test kit of claim 46 wherein the membrane comprises at least one material selected from nitrocellulose, cellulose, glass fiber, bonded glass fiber, polyesters, nylon, and polyethylsulphone.

60. The test kit of claim 46 further comprising a control line for confirming the device is working, the control line having an antibody to the labeled first antibody.

61. The test kit of claim 46 producing a test result within from about 0.5 to about 20 minutes after the sample is introduced to the porous membrane.

62. A test kit for rapid detection of pathogenic prion protein in a vertebrate, comprising:
(a) a buffer for extracting prion protein from a sample containing biological material obtained from a vertebrate;
(b) proteinase-K immobilized in a digestive pad for digesting the noninfectious prion protein in the sample;
(c) a test strip having an immobilized antibody of an antibody pair to the pathogenic prion protein, the test strip being in fluid communication with the digestive pad; and
(d) a labeled antibody to the pathogenic prion protein for producing a readable response indicative of the presence or concentration of the pathogenic prion protein.

63. The test kit of claim 62 wherein the test strip has pores of a diameter sufficient to allow the labeled antibody to migrate laterally through the test strip toward the immobilized antibody.

64. The test kit of claim 62 wherein the proteinase-K is covalently bound to the digestive pad.

65. The test kit of claim 62 wherein the proteinase-K is in a gelled substance contained in the digestive pad.

66. The test kit of claim 62 wherein the proteinase-K is immobilized on a support selected from latex beads, rod-shaped bodies coated with latex, micro- or nanoparticles, and a porous membrane pad.

67. The test kit of claim 62 further comprising a conjugate pad in fluid communication with the test strip, the labeled antibody being disposed in the conjugate pad.

68. The test kit of claim 62 wherein the proteinase-K is present in an amount ranging from about 30 μg to about 400 μg.

69. The test kit of claim 62 wherein the proteinase-K is present in an amount ranging from about 100 μg to about 350 μg.

70. The test kit of claim 62 wherein the labeled antibody has a colored label.

71. The test kit of claim 62 wherein the labeled antibody has a label selected from latex beads, rod-shaped bodies coated with latex, particles comprising a dye, colloidal particles, metal particles, micro- and nanoparticles, fluorescent compounds, chemiluminescent compounds, and magnetic beads.

72. The test kit of claim 67 further comprising a spacer pad between the digestive pad and the conjugate pad to allow for longer digestion of the nonpathogenic prion protein.

73. The test kit of claim 67 wherein the digestive pad and the conjugate pad comprise separate portions of a single pad.

74. The test kit of claim 62 wherein the digestive pad comprises at least one material selected from the group consisting of gauze, cellulose, cellulose acetate, polyesters, and porous materials.

75. The test kit of claim 62 wherein the conjugate pad comprises at least one of plastic filter bed in glass fiber, polyester, plastic bonded glass fiber and nonwoven polymeric materials.

76. The test kit of claim 62 wherein the test strip comprises at least one material selected from nitrocellulose, cellulose, glass fiber, bonded glass fiber, polyesters, nylon, and polyethylsulphone.

77. The test kit of claim 67 further comprising a control line for confirming the device is working, the control line comprising an antibody to the labeled first antibody.

78. A test device for detecting pathogenic prion comprising:
(a) proteinase-K immobilized on a support in an amount sufficient to substantially digest all noninfectious prion protein in a test sample;
(b) a labeled first antibody specific to the prion protein; and
(c) a membrane for lateral flow, having a first end, a second end, and second antibody to the prion protein immobilized therebetween; the first end being in fluid communication with the proteinase-K support and the labeled antibody;
such that the prion protein in an enzyme-treated sample migrates toward the second end of the membrane and binds with both of the antibodies to indicate the presence or concentration of prion protein in the test sample.
